(19) United States
(12) Patent Application Publication
(10) Pub. No.: US 2017/0143891 A1
(43) Pub. Date: May 25, 2017

(54) SYSTEMS AND METHODS FOR PRESSURE-REGULATED VOLUME CONTROL DURING CARDIOPULMONARY BYPASS AND PERFUSION PROCEDURES

(71) Applicant: Cardio-Myogen, LLC, Norristown, PA (US)

(72) Inventor: Charles R. Bridges, Newton, MA (US)

(73) Assignee: Cardio-Myogen, LLC, Norristown, PA (US)

(21) Appl. No.: 15/354,917

(22) Filed: Nov. 17, 2016

Related U.S. Application Data
(60) Provisional application No. 62/258,715, filed on Nov. 23, 2015.

(51) Int. Cl. A61M 1/36; A61M 1/16

(52) U.S. Cl. CPC A61M 1/3666; A61M 1/3638; A61M 1/3639; A61M 1/3659; A61M 1/367; A61M 1/1698; A61M 2205/3337; A61M 2205/8206; A61M 2205/8268; A61M 2205/50; A61M 2205/52; A61M 2205/3344; A61M 2205/3379

(57) ABSTRACT

Systems and methods for regulating fluid volume from an isolated cardiac circuit during cardiopulmonary bypass surgery are described. In one embodiment, a system in accordance with the present technology can include a pressure-regulated volume control unit configured to regulate a cardiac circuit volume based on a measured return pressure detected at an outflow from an internal heart portion of cardiac circuit. The system can also include a first pressure sensor configured to detect the measured return pressure.

### RELATED PATENTS INCORPORATED BY REFERENCE

U.S. Pat. No. 8,556,842, entitled “PERFUSION CIRCUIT AND USE THEREIN IN TARGETED DELIVERY OF MACROMOLECULES,” and U.S. Pat. No. 8,158,119, entitled “CARDIAC TARGETED DELIVERY OF CELLS,” are related to the present application, and the foregoing patents are incorporated herein by reference in their entireties. As such, components and features of embodiments disclosed in the patents incorporated by reference may be combined with various components and features disclosed and claimed in the present application.

### STATEMENT REGARDING FEDERALLY SPONSORED RESEARCH OR DEVELOPMENT

This invention was made with government support under grant R01-HL083078-05 awarded by the National Institutes of Health. The US government has certain rights in the invention.

### TECHNICAL FIELD

The present disclosure relates generally to systems and methods for pressure-regulated volume control during cardiopulmonary bypass and perfusion procedures. In particular, several embodiments are directed to systems and methods for regulating fluid volume from an isolated cardiac circuit during a cardiopulmonary bypass surgery, such as, for example, while therapeutically implementing a cardiac perfusion circuit for targeted delivery of therapeutics to cardiac tissue.

### BACKGROUND

Cardiovascular disease is a major health concern and the leading cause of deaths worldwide. While cardiovascular disease is a class of diseases that involve the heart, cardiovascular disease refers to any disease that affects the cardiovascular system, such as cardiac disease, vascular diseases of the kidney or brain, and peripheral arterial disease. The causes of cardiovascular disease are diverse. For example, cardiac diseases can include inherited autosomal recessive conditions (e.g., sarcoglycan deficiencies), X-linked cardiomyopathy (e.g., cardiomyopathy associated with Becker's muscular dystrophy), genetic cardiomyopathies or “idiopathic” heart failure, coronary ischemia, cancer (e.g., cardiac sarcomas and other neoplasms), and diseases of the heart valves (e.g., valvular stenosis, valvular insufficiency or regurgitation) among others. In many of these examples, therapeutic agents have been developed that may be useful for treating these medical conditions. However it is increasingly important that a physician or surgeon delivering such therapeutic agents is able to efficiently and accurately isolate the targeted tissue for effective delivery of the agents. This can be particularly relevant when the concentration of the agents at the target site cannot be safely or effectively achieved by introduction at a site in the body remote from the targeted tissue. Additionally, the physician may only want to treat a diseased portion of an organ or tissue and/or avoid treating healthy portions or other non-diseased organs.

In a particular example, gene therapy may provide promising new therapies for many cardiovascular-related medical conditions as vectors and therapeutic transgenes have been identified for treatment of heart failure due to genetic defects (e.g., X-linked defects, autosomal recessive defects) as well as heart failure due to environmental agents, injury, or other perturbations. However, challenges associated with developing techniques for targeting and efficiently delivering such therapeutics at the target tissue have yet to be overcome, limiting the applicability of gene therapy for the treatment of heart failure. Given the difficulties associated with effective and efficient delivery of therapeutic agents such as gene therapy agents, there remains the need for safe and effective devices and methods for delivery of therapeutic agents to targeted tissue, such as cardiac tissue.

### BRIEF DESCRIPTION OF THE DRAWINGS

FIG. 1 is a schematic illustration of a cross-sectional view of a heart depicting the major chambers, blood vessels, blood flow patterns and anatomical features of the heart.

FIG. 2 is a schematic illustration of a cross-section view of the heart shown in FIG. 1 and further depicting particular components of a cardiac delivery system used to stop and/or redirect blood flow during a cardiopulmonary bypass procedure in accordance with an embodiment of the present technology.

FIG. 3 is a schematic illustration of a recirculating perfusion circuit formed during a cardiopulmonary bypass procedure in accordance with an embodiment of the present technology.

FIG. 4 is a schematic illustration of a cardiac delivery system and method in accordance with an embodiment of the present technology.

FIG. 5 is a flow diagram illustrating a method for delivering therapeutic agents to targeted cardiac tissue during cardiopulmonary bypass surgical intervention in accordance with an embodiment of the present technology.

FIG. 6 is a flow diagram illustrating a method for removing volume from circulation within a cardiac perfusion circuit in accordance with an embodiment of the present technology.

FIG. 7 is a schematic block diagram illustrating computing system software modules and subcomponents of a computing device suitable to be used in the system of FIG. 4 in accordance with an embodiment of the present technology.

### DETAILED DESCRIPTION

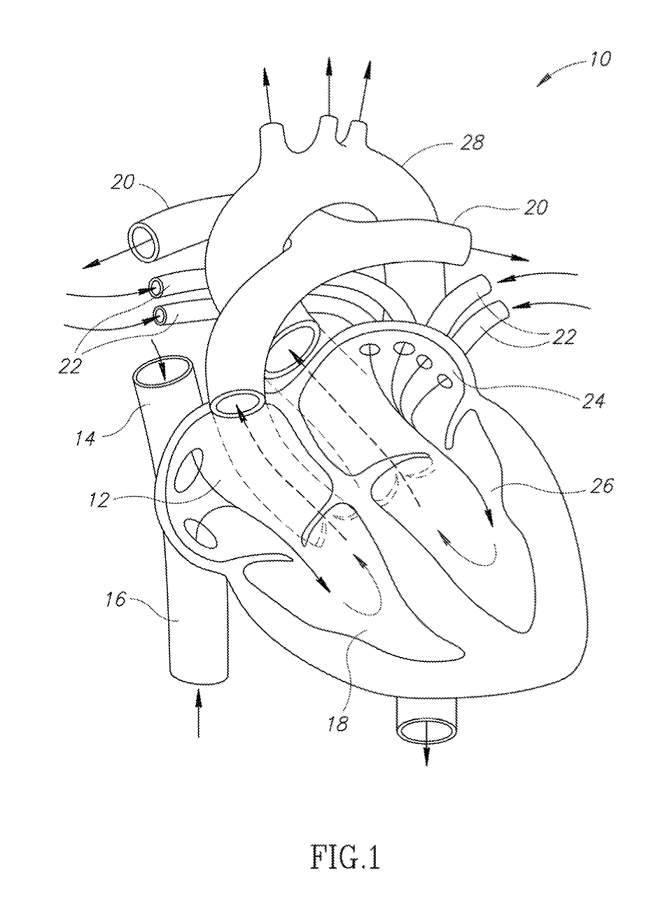

Specific details of several embodiments of the technology are described below with reference to FIGS. 1-7. Although many of the embodiments are described below with respect to devices, systems, and methods for surgical treatment and delivery of therapeutic agents to cardiac tissue for the treatment of heart diseases and conditions, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-7.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the technology. The headings provided herein are for convenience only and are not intended to limit or interpret the scope or meaning of the technology. For ease of reference, throughout this disclosure identical reference numbers are used to identify similar or analogous components or features, but the use of the same reference number does not imply that the parts should be construed to be identical. Indeed, in many examples described herein, the identically numbered parts are distinct in structure and/or function.

### A. OVERVIEW

The present disclosure describes pressure-regulated volume control systems for use with cardiac perfusion systems and devices, such as for example, for targeted delivery of therapeutic agents to cardiac tissue and associated methods. Several of the details set forth below are provided to describe the following examples and methods in a manner sufficient to enable a person skilled in the relevant art to practice, make and use them. Several of the details and advantages described below, however, may not be necessary to practice certain examples and methods of the technology. Additionally, the technology may include other examples and methods that are within the scope of the technology but are not described in detail.

Surgical methods and systems for implementing cardiac surgery with isolated, recirculating delivery of therapeutic agents are described. Certain embodiments of the present disclosure provide pressure-regulated volume control of fluid during recirculation within the cardiac circuit to prevent cardiac tissue distension. Aspects of the disclosure are further directed to systems, devices and associated methods that provide protection of non-targeted organs and tissues, by preventing leakage between the recirculating cardiac perfusion circuit and the systemically circulating blood volumes, thereby preventing the non-targeted tissues from exposure to the therapeutic agents.

Further aspects of the disclosure include therapeutic agents, compositions and formulations for use with devices and systems that enable isolated, recirculating delivery to cardiac tissue (e.g., for treatment and alteration of cardiac tissue without systemic exposure or dilution of the therapeutic agents, etc.). For example, effective delivery of gene therapy agents for use in treating various cardiac conditions may improve when concentrations of the agents are increased at the target site. Without being bound by theory, it is believed that the direct replacement of biologically important molecules in genetically deficient individuals is hampered by both (i) the inability of these molecules to reach intracellular sites and tissue sites such as cardiac tissue where they normally function and (ii) by the immunogenicity of these molecules.

Various aspects of the technology are directed to pressure-regulated volume control (PRVC) systems for use during cardiopulmonary bypass surgery. In one embodiment, the system can include a PRVC unit configured to regulate a cardiac circuit volume based on a measured return pressure detected at an outflow from an internal heart portion of a cardiac circuit. The system can also include a first pressure sensor configured to detect the measured return pressure. In various arrangements, the PRVC unit can be configured to remove a portion of perfusion solution from a fluid path formed by cardiopulmonary bypass when the measured return pressure exceeds a pre-determined return pressure value. In one example, the pre-determined return pressure value can be about 15 mm Hg. In certain embodiments, the PRVC unit can be configured to remove a minimum volume of a perfusion solution from a fluid path formed by cardiopulmonary bypass to reduce a return pressure from the measured return pressure to a desired return pressure.

In some embodiments, the pressure-regulated volume control systems can further include a controller in communication with the PRVC unit. The controller can have instructions, for example, that cause the PRVC unit to remove a portion of perfusion volume when the measured return pressure exceeds a maximum allowable return pressure. In other embodiments, the controller can be in communication with the PRVC unit and the first pressure sensor. The controller can be include instructions that are executable to (a) receive the measured return pressure from the first pressure sensor in communication with the outflow from the internal heart portion of the cardiac circuit, (b) calculate a minimum amount of volume of perfusion solution to remove from the cardiac circuit to reduce a return pressure from the measured return pressure to a desired return pressure, and (c) command the PRVC unit to remove the minimum amount of volume.

Other embodiments of the present technology are directed to systems for regulating fluid volume from an isolated cardiac circuit during cardiopulmonary bypass surgery. In one embodiment, the system can include a controller and a PRVC unit in communication with the controller. In certain embodiments, the PRVC unit can be configured to regulate a cardiac circuit fluid volume based on a measured return pressure detected at an outflow from an internal heart portion of a cardiac circuit. The controller can have instructions that cause the PRVC unit to measure a return pressure at the outflow of the internal heart portion and to remove a calculated minimum amount of fluid volume from the cardiac circuit.

Other aspects of the present technology are directed to methods for removing excess volume of fluid from circulation within a cardiac circuit during cardiopulmonary bypass in a subject. In one embodiment, the method can include measuring a return pressure at an outflow of a heart in the patient and comparing the measured return pressure to a desired return pressure. The method can also include calculating an amount of volume of fluid to remove from the cardiac circuit to achieve at least the desired return pressure if the measured return pressure is greater than the desired return pressure. The method can further include removing the calculated amount of volume of fluid. In some embodiments, removing the calculated amount of volume of fluid includes continuously removing a volume of fluid at a rate of removal between a specified maximum and minimum rate of removal. Additional embodiments of the method can further include maintaining the return pressure at or near the desired return pressure by altering the rate of removal. The calculated amount of volume of fluid can be removed and deposited, in some examples, into a waste reservoir. In additional embodiments, the total amount of volume of fluid removed from the cardiac circuit (e.g., during cardiopulmonary bypass) can be determined to not exceed a predetermined maximum about of volume of fluid (e.g., approximately 1000 ml).

Additional aspects of the technology are directed to cardiac delivery systems for selectively delivering a therapeutic agent to cardiac tissue during cardiopulmonary bypass surgery. In one embodiment, the system can include a fluid path through the heart formed by cardiopulmonary bypass. The path can provide an isolated cardiac circuit having an internal heart portion and an external circuit portion. The system can also include a first pump in fluid communication with the path, the first pump configured to control a flow rate of a perfusion solution through the path. The system can further include a second pump in fluid communication with the path and with a perfusion solution reservoir. In certain arrangements, the second pump can be configured to control a flow of perfusion solution which may incorporate oxygenated blood along with a perfusion solution (e.g., blood cardioplegia) from the perfusion solution reservoir into the path. The system can still further include a PRVC unit in communication with the path. The PRVC unit can be configured to regulate a cardiac circuit volume based on a measured return pressure detected at an outflow from the internal heart portion of the cardiac circuit.

Other embodiments of the present technology are directed to systems for delivering a therapeutic agent to cardiac tissue of a patient during cardiopulmonary bypass. In one embodiment, the system can include a fluid path through the heart formed by cardiopulmonary bypass. The path can provide an isolated cardiac circuit having an internal heart portion and an external circuit portion. The system can also include a first pump in fluid communication with the path, the first pump configured to control a flow rate of a perfusion solution through the path. The system can further include a controller and a PRVC unit in communication with the path. The PRVC unit can be configured to regulate a cardiac circuit volume based on a measured return pressure detected at an outflow from the internal heart portion of the cardiac circuit. In some arrangements, the controller has instruction that cause the PRVC unit to (a) measure a return pressure at an outflow of the internal heart portion, and (b) remove a calculated minimum amount of volume of perfusion solution from the cardiac circuit. In one embodiment, the controller has instruction that are executable to (1) compare the measured return pressure to a pre-determined maximum return pressure, and (2) calculate a minimum amount of volume of perfusion solution to remove from the cardiac circuit to achieve at least the pre-determined maximum return pressure.

### B. SELECTED EMBODIMENTS OF SYSTEMS FOR ISOLATION OF CARDIAC AND SYSTEMIC CARDIOPULMONARY BYPASS CIRCUITS

The human heart is a muscular organ that provides continuous blood circulation through the cardiac cycle. The heart can be divided into four main chambers called the right and left atria and the right and left ventricles. The right heart, contains the right atrium and ventricle, and is separated by two muscular walls or septa (i.e., atrial septum and ventricular septum) from the left heart, containing the left atria and ventricle. The right heart supplies the lung (pulmonary) circulation while the left heart supplies the remaining circulation to the body. To insure that blood flows in one direction from the right to the left heart, atrioventricular valves are present at the inlet junctions of the atria and the ventricles (the tricuspid valve on the right and the mitral valve on the left), and semi-lunar valves (the pulmonary valve on the right and the aortic valve on the left) govern the exits of the ventricles leading to the lungs and the rest of the body. These valves contain leaflets that open and shut in response to blood pressure changes caused by the contraction and relaxation of the heart chambers. FIG. 1 is schematic illustration of a cross-sectional view of a heart 10 depicting the major chambers, blood vessels, blood flow patterns and anatomical features of the heart 10.

Referring to FIG. 1 and with respect to blood flow patterns, oxygen-poor blood is returned from the body to the right atrium 12 of the heart 10 via two large veins, the superior vena cava 14 and the inferior vena cava 16. From the right atrium 12, the blood is pumped into the right ventricle 18 and then to the pulmonary artery 20 before passing to the lungs (not shown) where the blood is oxygenated. Oxygen-rich blood returns from the lungs via four pulmonary veins 22 into the left atrium 24, and is subsequently pumped into the left ventricle 26. Upon contraction of the left ventricle 26, the oxygenated blood flows into the aorta 28 where it is circulated throughout the body. Coronary arteries (not shown) connect to the aorta 28 and provide oxygen-rich blood to the heart. A network of coronary veins (not shown) returns the oxygen-poor blood utilized by the heart into the right atrium 12 via the coronary sinus (not shown).

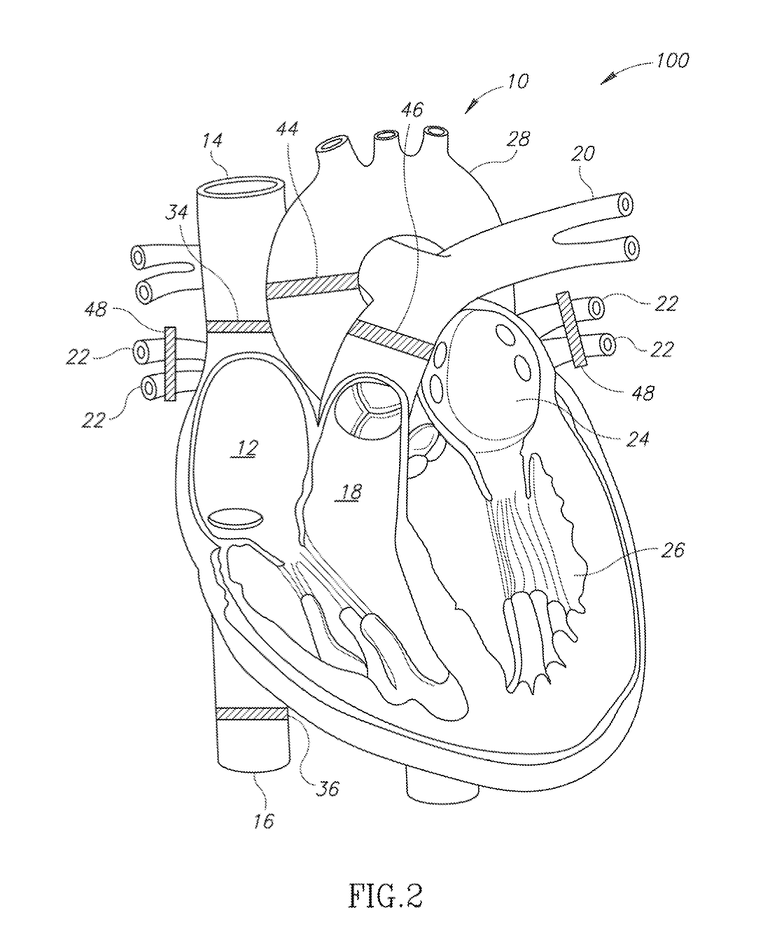

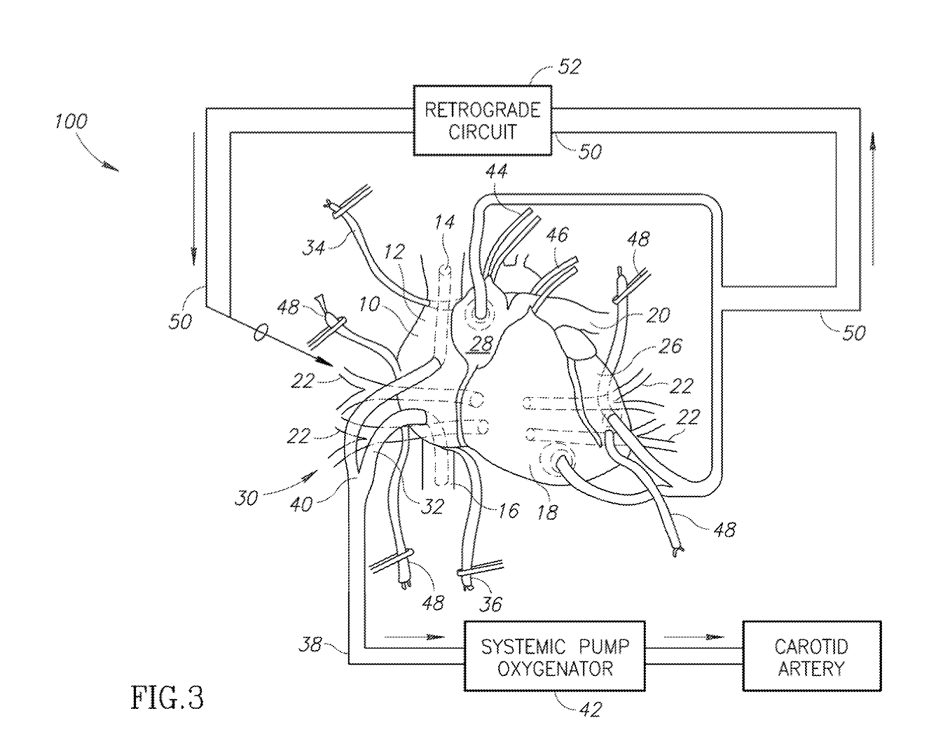

The heart can be isolated in situ via the formation of separate cardiopulmonary bypass circuits for cardiac and systemic circulation. FIG. 2 is a schematic illustration of a cross-section view of the heart 10 shown in FIG. 1 and further depicting particular components of a cardiac delivery system 100 that are used to stop and/or redirect blood flow during a cardiopulmonary bypass procedure and FIG. 3 is a schematic illustration of a recirculating perfusion circuit formed during a cardiopulmonary bypass procedure, which are in accordance with embodiments of the present technology.

Referring to FIGS. 2 and 3 together, the present technology provides a cardiac delivery system 100 which includes devices and components for achieving in situ isolation of the heart from the systemic circulation. The cardiac delivery system 100 is suitable for forming a systemic cardiopulmonary bypass circuit 38 isolated (e.g., separated) from a cardiac perfusion circuit 50 such that therapeutic agents can be effectively and safely delivered to the cardiac tissue within the cardiac perfusion circuit 50 and while continuing to deliver oxygenated blood to the systemic tissue during surgery (FIG. 3). For such surgical cardiopulmonary bypass procedures, and as shown in FIGS. 2 and 3, the system 100 can include venous cannulae with or without right angle tips, 30 and 32, that are surgically positioned within the superior vena cava 14 and the inferior vena cava 16 for redirecting oxygen-poor blood returning from the body away from the normal blood flow through the heart 10 as described above (FIG. 1). Snares (e.g., clamps), 34 and 36, are placed about the superior vena cava 14 and the inferior vena cava 16, respectively, to hold the cannulae 30, 32 in position, prevent systemic blood leakage into the cardiac perfusion circuit 50 and so that all systemic venous return flows into the systemic cardiopulmonary bypass circuit 38 via a Y-connector 40. In various arrangements, the systemic circuit 38 can include an oxygenator 42 (e.g., a pump oxygenator, heart-lung machine, cardiopulmonary bypass pump, etc.), or like mechanism, and can return oxygen-rich blood to the subject's femoral and/or carotid arteries via a cannula (not shown). The aorta 28 and pulmonary artery 20 are cross-clamped with clamps 44 and 46, respectively, to further isolate cardiac circulation from systemic circulation.

In some embodiments, all for pulmonary veins 22 are closed with snares (e.g., clamps) 48 such that cardiac circulation is isolated from systemic circulation and systemic circulation is isolated from cardiac circulation (e.g., two-way isolation). As described herein, the isolation of cardiac circulation from systemic circulation provides a shorter, more concentrated fluid circuit (e.g., less volume) for delivery of therapeutic agents to the heart and further permits additional re-circulation of the therapeutic agents through the circuit during cardiopulmonary bypass (CPB) to increase delivery effectiveness of the therapeutic agents.

Referring to FIGS. 2 and 3 together, and in one embodiment, the cardiac perfusion circuit 50 can be a retrograde circuit (e.g., cardiac circulation proceeding in a direction opposite of normal blood flow as shown in FIG. 1). For example, FIG. 3 illustrates the fluid flow path for retrograde perfusion via the coronary sinus. As shown, the path permits multi-pass retrograde re-circulation of therapeutic agents in solution through the two way isolated cardiac perfusion circuit 50 within the heart 10. In a particular example, and without being bound by theory, it is believed that since the capillaries lie on the venous side of the arteriolar resistor, retrograde (e.g. venous to arterial) vector infusion results in a higher capillary to interstitial pressure gradient favoring filtration of the therapeutic agent (e.g., vector plus transgene). Since endothelium can be rate-limiting for delivery of therapeutic agents such as macromolecular molecules (e.g., vector-mediated gene transfer), it is believed that a retrograde approach results in enhanced transfection efficiency. However, in other embodiments, the fluid flow path can be antegrade (e.g., cardiac circulation proceeding in a normal blood flow direction as shown in FIG. 1), or in a combination of retrograde and antegrade perfusion.

The cardiac perfusion circuit 50 includes the portion of the circuit that flows through the heart chambers and vessels, and also includes an exterior portion 52 of the circuit (FIG. 3). In particular, the cardiac delivery system 100 includes a number of devices and components that enhance the safety of the cardiac isolation procedure that reside outside of the portion of the circuit that flows through the heart 10.

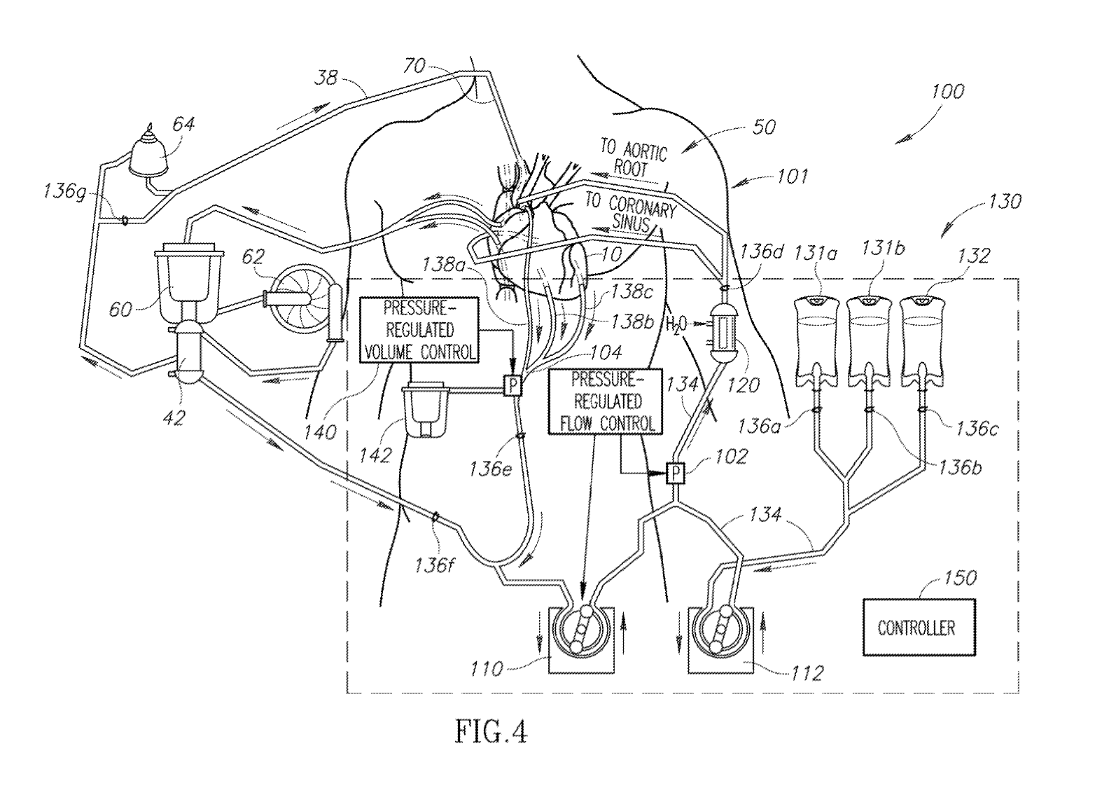

FIG. 4 is a schematic illustration of the cardiac delivery system 100 implemented in a human patient 101 in accordance with one embodiment of the present technology. As illustrated in FIG. 4, the system 100 provides for the systemic cardiopulmonary bypass circuit 38 and the cardiac perfusion circuit 50 for efficacious delivery of therapeutic agents to targeted cardiac tissue. In various embodiments, the system 100 incorporates devices (described in further detail herein) for pressure-regulated flow control (PRFC). For example, the system 100 can monitor a cardiac circuit inflow pressure at the in-flow 102 to the coronary sinus (e.g., in a retrograde format) and/or at the tip of the coronary sinus catheter (not shown) and adjust a flow rate of the perfusion solution in response to the inflow pressure. In some embodiments, the system 100 further incorporates a pressure-regulated volume control (PRVC) unit 140 (described in further detail herein) for auto-regulating a cardiac circuit volume based on a return pressure monitored at the outflow 104 from the heart 10.

To establish the cardiac perfusion circuit 50 and recirculation of the desired solutions to the cardiac tissue, the system 100 includes first and second cardiac circuit pumps 110, 112, a heat exchanger 120, a perfusion solution reservoir station 130 for holding delivery solution reservoirs for cardioplegia solutions 131 (shown individually as 131a and 131b) and flush solution(s) 132, and tubing 134 for circulating such delivery and perfusion solutions (or other solutions) in circuit pathways exterior to the heart 10. The system 100 can further incorporate a plurality of controllable switches 136 or valves (shown individually as switches/valves 136a-136g) for auto-regulating directionality, volume and flow rate of perfusion solutions through the cardiac perfusion circuit 50 and/or blood through the systemic circuit 38. For example, particular switches/valves 136 (described further herein), can be utilized to direct flow for delivery of cardioplegia solutions 131 or to recirculate a therapeutic agent through the heart 10. Accordingly switches/valves 136 can be activated depending on the application.

In one embodiment, the exterior portion 52 (FIG. 3) of the cardiac circuit 50 includes the first or master pump 110 (e.g., a rotary pump, a roller pump, etc.) for controlling the rate of circulation of perfusion solution (e.g., the therapeutic agent solution) through the circuit 50. The system 100 can also include the second or follower pump 112 (e.g., a rotary pump, a roller pump, etc.) for controlling a volume and flow rate of delivery solutions 131a, 131b, 132 to be supplied to the cardiac perfusion circuit 50.

In reference to FIG. 4, and in operation, a first volume of oxygenated blood from the systemic circuit oxygenator 42 can be diverted through an open valve 136f, passed through the first pump 110 and mixed (e.g., in a 4:1 ratio or other ratio) with a second volume of delivery solution (e.g., cardioplegia) delivered from the perfusion solution reservoir station 130 and through the second pump 112. The pressure of the mixed perfusion solution can be monitored at the in-flow 102 (e.g., via a pressure sensor) before passing through the heat exchanger 120, and optionally an oxygenator (not shown) to allow control of the temperature and oxygen content of the perfusion solution being circulated through the cardiac circuit 50. When the circuit flows in retrograde, the perfusion solution passes through a cardioplegia switch 136d open to the coronary sinus (e.g., switch 136d is closed to the aortic root) and through the heart 10. Perfusion solution can exit the heart aorta, the right ventricle and the left ventricle through vent catheters 138a-c. In other embodiments, not shown, the perfusion solution can exist through the pulmonary artery or pulmonary veins. At the outflow 104, the PRVC unit 140 can assess return pressure (e.g., via a pressure sensor). If the return pressure exceeds a pre-established maximum pressure (e.g., about 15 mm Hg to about 20 mm Hg) and/or pressure range, the PRVC unit 140 can draw off a portion of the perfusion solution to reduce a volume circulating within the cardiac circuit 50. The drawn off portion can be directed into a waste reservoir 142.

As shown in FIG. 4, the systemic cardiopulmonary bypass circuit 38 provides a blood flow circuit to the rest of the body 101. Venous blood redirected from the superior and inferior vena cava 14, 16 (FIGS. 1-3) via cannulae 30 and 32, direct the oxygen-poor blood to a venous reservoir 60. From the venous reservoir 60, the oxygen-poor blood is pumped via systemic pump 62 (e.g., centrifugal pump, rotary pump, roller pump, etc.) to the oxygenator/heat exchanger 42. Oxygen-rich blood can optionally be directed through a filter 64 for removal of air bubbles and/or particulates by the closure of valve 136g. Oxygen-rich blood is pumped into the carotid artery via cannulation 70 where it circulates through the body 101. In other configurations, not shown, oxygen-rich blood can be pumped into the ascending aorta or femoral artery for distribution through the body 101.

The system can further include a controller 150 in communication with any or all other components of the system 100, including the cardiac perfusion circuit 50 components as well as the systemic circuit 38 components. In one embodiment, the controller 150 has instructions for causing the release of delivery solutions (e.g., cardioplegia, flush, or other solutions) to the heart 10 or to recirculate the solutions (e.g., containing therapeutic agents) through the heart. For example, the controller 150 can direct fluid direction and redirection by controlling activity of the switches/valves 136. In some embodiments, the controller 150 can include instructions for monitoring PRFC and PRVC and taking corrective action if pressure measurements fall outside of allowable parameters. In other embodiments, the controller 150 can include instructions for halting, slowing or otherwise altering fluid flow through the pumps 110, 112 or 62 if bubbles (e.g., due to air) are detected in the circuit and/or initiating an alarm. In one example, if pressure monitored at the outflow 104 from the heart 10 exceeds a maximum allowable pressure (e.g., about 15 mm Hg), the PRVC unit 140 can be engaged by the controller 150 to remove an appropriate amount of volume to the waste reservoir 142. Likewise, inflow pressure can be monitored at inflow 102 and the controller 150 can be configured to increase or decrease a flow rate (e.g., via first pump 110) such that a maximum allowable flow rate is achieved without exceeding a maximum in-flow pressure (e.g., about 40 mm Hg to about 100 mm Hg) while keeping the minimum in-flow pressure above a given threshold (e.g., about 30 mm Hg to about 80 mm Hg). The controller 150 can further include a user interface (not shown) for receiving parameters from an operator before and/or during operation.

Generally during cardiopulmonary bypass procedures, blood leakage from the cardiac circuit 50 to the systemic circuit 38 is minimal or not present. Having the cardiac circuit 50 isolated from the systemic circulation is important as such leakage could cause therapeutic agents (e.g., virus) to spill into the systemic circulation and increase the probability of a) an immune response against the therapeutic agent (e.g., viral capsid); collateral organ exposure (e.g., gene transfer/gene expression, such as gonadal gene transfer, and c) could decrease the dose of therapeutic agent (e.g., virus) administered to the heart. However, during cardiopulmonary bypass procedures, the present inventors discovered that there can be some leakage of spillover of blood from the systemic circuit 38 to the cardiac circuit 50 (e.g., non-complete or “one-way” isolation). Such leakage of blood from the systemic circuit 38 to the cardiac perfusion circuit 50 may cause the left and right ventricular cavities to progressively distend under progressively higher pressures during intervals of recirculation of the perfusion solutions (e.g., containing therapeutic agent). For example, return pressure measured at the outflow 104 (FIG. 4) of the heart 10 can rise progressively during recirculation such that the return pressure (which starts at zero at the start of the perfusion activity) can increase to about 20 mm Hg within approximately five minutes. This return pressure is also communicated to the left ventricular and right ventricular cavities and to the pulmonary veins that drain into the left ventricle. As recirculation of perfusion solutions can occur for about 10 minutes to about 20 minutes in some embodiments, the progressive increase in return pressure can lead to dysfunction of the left and right ventricles and can further lead to pulmonary congestion and lung dysfunction.

To address these short-comings, the present disclosure provides embodiments of the cardiac delivery system 100 including the PRVC unit 140, which can optimally remove a portion of volume of the perfusion solution in one or more cycles in a manner that automatically reduces pressure to a desired level or within a desired range. The system 100 can also be configured to have the PRVC unit 140 remove as little volume of perfusion solution as possible to minimize the loss of therapeutic agent(s). For example, if the therapeutic agent is a virus (e.g., for gene therapy applications), a high concentration of the virus during recirculation is important for efficacious transfer/delivery of the therapy to the cardiac tissue. Hence, reduction of a concentration of the therapeutic agent is minimized using the PRVC unit 140.

The controller 150, in some embodiments, stores and executes instructions for commanding the PRVC unit 140 to monitor and remove volume from the cardiac perfusion circuit 50. In some embodiments, the PRVC unit 140 may receive instructions to monitor and/or remove excess volume (e.g., to reduce the return pressure to or below a desired level) at particular time intervals, or in other embodiments on a continuous basis. In another embodiment, the controller 150 includes instructions that are executable to a) receive a measured return pressure value from the PRVC unit 140 (e.g., from a pressure sensor within unit 140) or from a separate pressure sensor (not shown) near the outflow 104, b) calculate a minimum amount of volume of perfusion solution to remove from the cardiac perfusion circuit 50 in order to achieve a desired return pressure within an acceptable range (e.g., below a maximum pressure value, at or below about 15 mm Hg, etc.), and c) command the PRVC unit 140 to remove the minimum amount of volume.

The controller 150 can also be programmed to control the PRVC unit 140 such that the PRVC unit delivers the removed perfusion solution to the waste reservoir 142. The rate of fluid removal can be based on, for example, processing information (e.g., protocol, pre-determined pressure parameters, predetermined concentration of therapeutic agent(s), procedure time(s), recirculation cycles, etc.), flow rate information (e.g., flow rates under certain conditions, the actual flow rate for a certain type of perfusion solution, etc.), or can be individualized based on the arterial-alveolar oxygen gradient in a given patient. For example, a patient whose lungs are having difficulty maintaining adequate oxygenation, the maximum pressure value could be set to a lower threshold (e.g., 10 mm Hg), and the like. In some embodiments, the volume of the perfusion solution to be removed can be determined based on an initial volume of perfusion solution within the cardiac perfusion circuit 50 and an estimated rate of increase of volume per cycle. The stored flow rates can be input into the system 100 or determined by the system 100. In some embodiments, the controller 150 can calculate an equilibrium volume (e.g., desired circulation volume with a safe return pressure value) in advance (e.g., a pilot run or preliminary run prior to delivery of therapeutic agent), and the system 100 can use the determined equilibrium volume as the initial volume for the same kind of perfusion solutions. In some embodiments, the equilibrium volume infused into the heart is in the range of about 20%/o to about 100%, about 25% to about 90%, about 30% to about 80%, about 40% to about 70%, about 50% to about 60% of the estimated volume of the patient's heart.

Then the controller 150 can instruct the first and/or second pumps 110, 112 to provide the oxygen-rich blood and delivery solution(s), respectively, at a rate (e.g., a rate determined by the pilot run). In some embodiments, the infusion can be generally over about 30 seconds to about 1 minute at a circuit flow rate of about 40 cc/min to 140 cc/min, or about 80 cc/min to about 120 cc/min. The flow direction, the circuit flow speed, and the circuit interval frequency can be adjusted depending on the type of perfusion solutions, the particular characteristics of the therapeutic agents and the desired recirculation interval profile. In one example, if the surgical protocol calls for 15 minutes of recirculation time for perfusion of a solution infused with therapeutic agent, and the circulation volume is maintained at about 100 cc, the system 100 will provide for about 15 intervals for the therapeutic agent to flow through the heart at flow rate of about 100 cc/min.

A power source (not shown) of the controller 150 can be electrically coupled to the controller 150 as well as other components of the system 100 (e.g., pumps 110 and 112, heat exchanger 120, etc.). The power source can be one or more batteries, fuel cells, or the like. The power source can also deliver electrical energy to other components of the system 100 within the systemic cardiopulmonary bypass circuit 38. In other embodiments, the power source can be an AC power supply.

### C. SELECTED EMBODIMENTS OF CARDIAC PERFUSION METHODS

The system 100 can be used to perform several cardiopulmonary bypass and treatment delivery methods. Although specific examples of methods are described herein, one skilled in the art is capable of identifying other methods that the system 100 could perform. Moreover, the methods described herein can be altered in various ways. As examples, the order of illustrated logic may be rearranged, sub-stages may be performed in parallel, illustrated logic may be omitted, other logic may be included, etc.

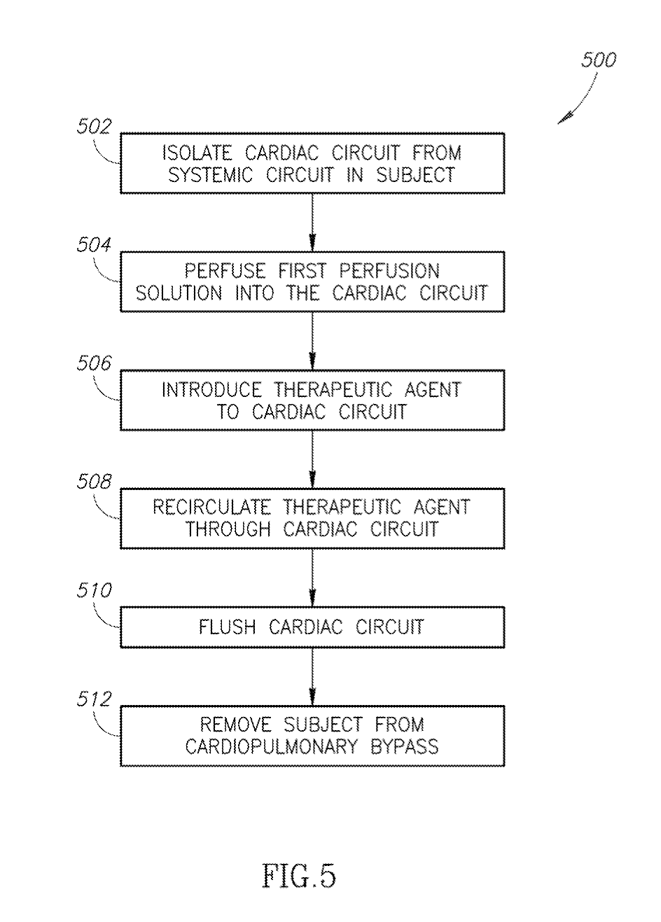

FIG. 5 is a flow diagram illustrating a method 500 for delivering therapeutic agents to targeted cardiac tissue during cardiopulmonary bypass surgical intervention in accordance with embodiments of the present technology. Even though the method 500 is described below with reference to the cardiac delivery system 100 of FIGS. 3 and 4, the method 500 may also be applied in other treatment systems with additional or different hardware and/or software components.

As shown in FIG. 5, an early stage of the method 500 can include isolating a cardiac circulation circuit from a systemic circulation circuit in a subject using a cardiopulmonary bypass procedure (block 502). In a particular example, the procedure can include the following steps: (a) the subject is cannulated (e.g., in the left femoral artery) for blood pressure monitoring, (b) the aorta and pulmonary artery are ensnared using umbilical tapes, (c) the pulmonary artery is ensnared by exclusion, and (d) the right carotid artery is cannulated. In continuing this example and using previously placed purse strings: 1) a cardioplegia cannula (containing a vent limb) is placed in the ascending aorta; 2) the superior vena cava is cannulated; 3) a retrograde catheter is placed into the coronary sinus and 4) the inferior vena cava is cannulated. The two venous cannulae are connected to a Y connector and connected to the venous limb of the systemic pump circuit. Cardiopulmonary bypass (CPB) is initiated. In this example, all of the pulmonary veins are ensnared, individually or in groups using umbilical tapes, tourniquets or other snares known by those in the relevant surgical field. The azygous vein can be ligated. Following the inferior vena cava is snared, and a cannula is placed into the left ventricular cavity and clamped. A cannula can then be placed into the right ventricle and clamped and the purse string suture can be snared. Accordingly, following block 502, the cardiac circuit, illustrated schematically in FIG. 3, is constructed and the cardiac perfusion circuit is isolated from the systemic cardiopulmonary bypass circuit. In various embodiments, systemic cooling to approximately 15° C. to about 34° C., and in a particular embodiment, to about 32° C., can be initiated. The cardiac circuit is isolated and the heart can be emptied of excess volume and air. Additionally, components, systems and method for forming cardiopulmonary bypass in a subject is described in commonly assigned U.S. Pat. Nos. 8,158,119 and 8,556,842 and in Bridges et al., Annals of Thoracic Surgery, 73:1939-1946 (2002), which is incorporated by reference in its entirety.

The method 500 can also include perfusing a first perfusion solution into the cardiac circuit in a retrograde direction (block 504). Flow of the first perfusion solution into the isolated cardiac circuit is continued until the coronary sinus pressure is between about 40 mm Hg to about 80 mm Hg. In one embodiment, the flow rate of the first perfusion solution is about 80 mL/min to about 150 mL/min. In some embodiments, the first perfusion solution can be a pretreatment solution (e.g., a solution not containing a therapeutic agent). For example, the first perfusion solution can be an albumin solution (e.g., human serum albumin) which may pre-treat the cardiac circuit pathway prior to delivery of a therapeutic agent by decreasing the likelihood of inactivation of the therapeutic agent. In one example, it has previously been demonstrated that adenoviral vector can be inactivated upon contact with the materials which form a perfusion catheter and that human serum albumin pre-treatment can prevent this inactivation.

In another embodiment, the first perfusion solution can include a vascular permeability-enhancing agent. For example, in some embodiments, it can be useful to use a vascular permeability-enhancing agent prior to delivery of certain therapeutic agents, to enhance, for example, uptake of the therapeutic agents into the targeted cardiac tissues. Some examples of vascular permeability-enhancing agents include, e.g., nitroglycerine, histamine, acetylcholine, an adenosine nucleotide, arachidonic acid, bradykinin, cyanide, endothelin, endotoxin, interleukin-2, ionophore A23187, nitroprusside, a leukotriene, an oxygen radical, phospholipade, platelet activating factor, protamine, serotonin, tumor necrosis factor, vascular endothelial growth factor, a venom, and a vasoactive amine. In some embodiments, vascular permeability-enhancing agents are not used or are unnecessary for effective delivery of the therapeutic agent(s). Alternatively, if vascular permeability-enhancing agents are used, in certain embodiments, they may be infused simultaneously with the therapeutic agent(s).

In other embodiments, the first perfusion solution can include other compositions such as cardioplegic solution (e.g., Plegisol® cardioplegic solution), or other drugs, gene therapies and/or medication.

At block 506, the method 500 includes introducing a therapeutic agent in solution to the cardiac circuit. In some embodiments, the first perfusion solution is removed by the system prior to introducing the therapeutic agent solution. In other embodiments, the therapeutic agent is infused (e.g., in high concentration) into the circulating solution such that the volume of first perfusion solution and the infused therapeutic agent solution is a desired circulating volume for the treatment of the heart. The therapeutic agent solution can be, in some embodiments, introduced at about 0.1 mL/kg to about 3 mL/kg, or at about 0.5 mL/kg. The desired circulating volume within the cardiac circuit can be in the range of about 20% to about 100%, about 25% to about 90%, about 30% to about 80%, about 40% to about 70%, about 50% to about 60% of the estimated volume of the subject's heart. In some embodiments, introduction of the therapeutic agent solution can be slow and can be infused over about 30 seconds to about 1 minute at a circuit flow rate of about 80 cc/min. to about 140 cc/min., or at about 100 cc/min. to about 120 cc/min. In one embodiment, the therapeutic agent in solution is introduced and circulated in a retrograde flow direction. In another embodiment, the method 500 can include delivering the therapeutic agent in solution simultaneously in both the retrograde (through the coronary sinus, FIG. 4) and antegrade direction (through aortic root, FIG. 4) during cardiac isolation.

The method 500 can further include recirculating the therapeutic agent in solution through the cardiac circuit (block 508). In some embodiments, recirculation continues for approximately 30 minutes. In other embodiments, recirculation occurs for less than 30 minutes (e.g., about 25 minutes, about 20 minutes, about 15 minutes, about 10 minutes, etc.). In other embodiments, circulation is stopped and the solution is allowed to dwell for about 30 seconds to about 10 minutes, or about 1 minute to about 9 minutes, or about 2 minutes to about 5 minutes. If circulation is halted, flow can be restored over, for example, one minute to about 50 cc/min. to about 120 cc/min., with coronary sinus pressure equal to about 40 mm Hg to about 80 mm Hg. In various arrangements, an additional volume of therapeutic agent and/or a volume of a second therapeutic agent can be infused to the cardiac circuit upon restarting the circuit flow after a dwell period. In other embodiments, block 508 does not include a dwell time (i.e., the circulation is not stopped), and in such instances, recirculation of the therapeutic agent in solution can occur for the entire desired time (e.g., about 20 min, about 10 minutes, etc.).

In additional steps, the method 500 can include flushing the cardiac circuit (block 510) and removing the subject from cardiopulmonary bypass (block 512). In these steps, the coronary sinus catheter can be removed and the suture tied. The cardiac circuit is then flushed (block 510). In one embodiment, the perfusion solution reservoir station 130 contains the flush solution 132 (FIG. 4), which can be delivered using conventional techniques for flushing the perfusion solutions containing the therapeutic agent out of the cardiac circuit. In a particular example, some constituents of a flush solution can include Hespan, Benadryl, Solumedrol, and Zantac. Where the infusion has been retrograde, the cardiac circuit is generally flushed in an antegrade flow direction. An antegrade flush can include infusion of a suitable flush solution via the aortic route (e.g., the ascending aorta). Conventional techniques for removing the subject from cardiopulmonary bypass can be utilized (see, e.g., Bridges et al., Annals of Thoracic Surgery, 73:1939-1946 (2002)) and rewarming is initiated.

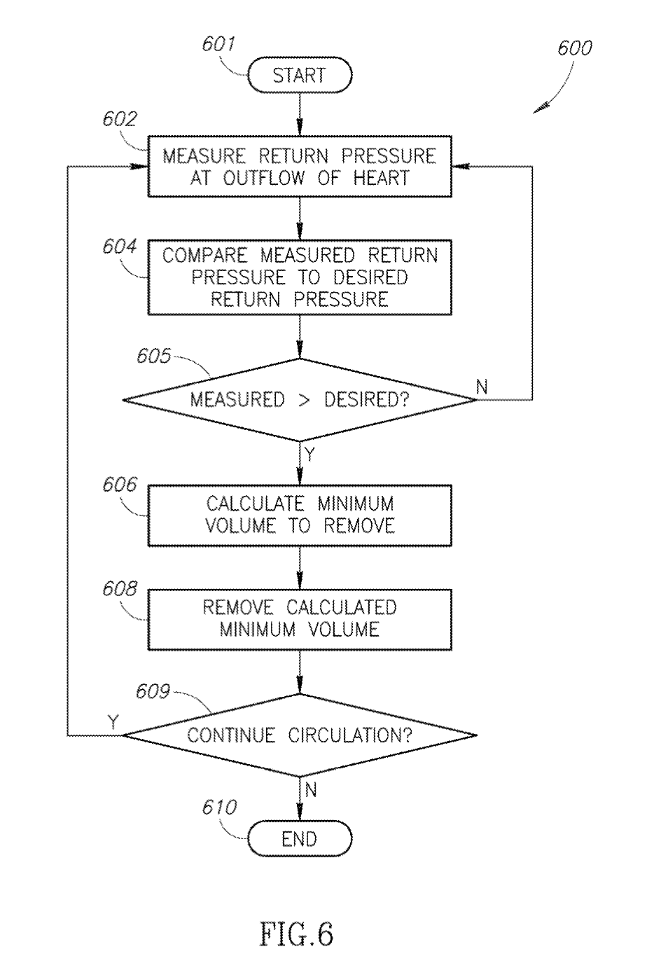

FIG. 6 is a block diagram illustrating a method 600 for removing excess volume from circulation within cardiac perfusion circuit 50 using the cardiac delivery system 100 described above and with reference to FIGS. 3 and 4. With reference to FIGS. 3, 4 and 6 together, the method 600 starts at 601 and can include measuring a return pressure at an outflow 104 (FIG. 4) of the heart 10 (block 602) and comparing the measured return pressure to a desired return pressure (block 604). In one embodiment the return pressure can be measured using a pressure sensor (not shown) at the outflow 104. In some embodiments, the pressure sensor can be associated with the PRVC unit 140 (FIG. 4). Referring to decision block 605, if the measured return pressure is at or below the desired return pressure, the method 600 can continue at block 602 (e.g., in interval patterns or continuously).

If the measured return pressure is greater than the desired return pressure (decision block 605), the method 600 can include calculating an amount of volume of perfusion solution to remove from the cardiac perfusion circuit 50 (FIG. 4) to achieve at least the desired return pressure (block 606). In one embodiment the calculated amount of volume to be removed in a minimum amount of volume. The method 600 can continue at block 608 with removing the calculated amount of volume. In one embodiment, the calculated amount of volume can be removed by the PRVC unit 140 in an automated manner and deposited in the waste reservoir 142 (FIG. 4). In other embodiments, the calculated amount of volume can be removed manually (e.g., by syringe used by an operator of the system 100). After the removal of the calculated amount of volume, the method 600 can return to block 602 if recirculation of the perfusion solution is to continue (decision block 609). If at decision block 609, the recirculation of the perfusion solution is to stop, the method 600 can end (block 610).

### D. SUITABLE COMPUTING ENVIRONMENTS

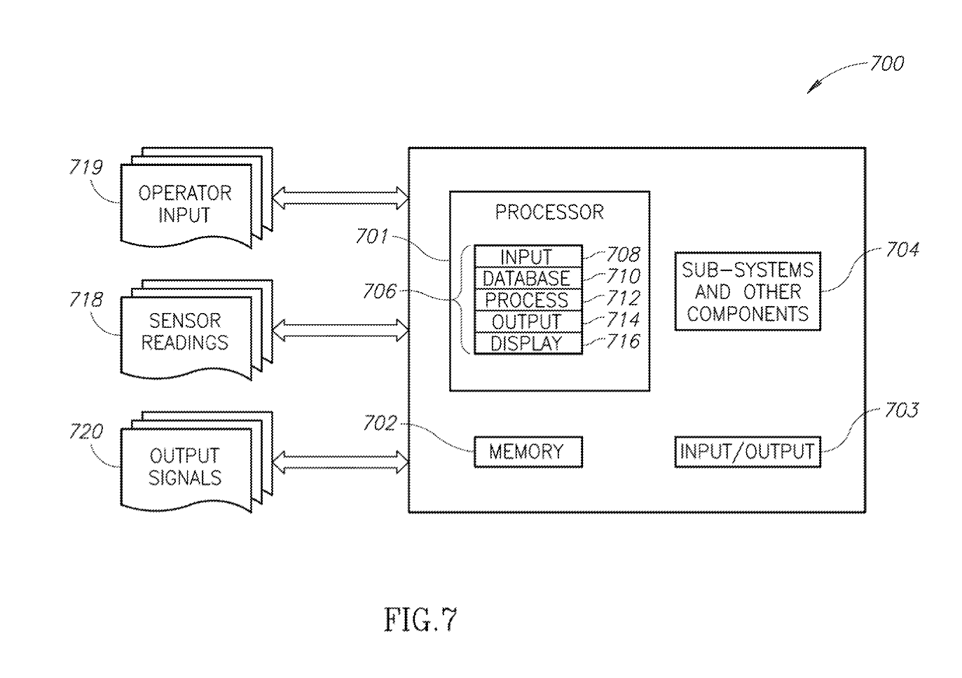

FIG. 7 is a schematic block diagram illustrating subcomponents of a computing device 700 in accordance with an embodiment of the present technology. The computing device 700 can include a processor 701, a memory 702 (e.g., SRAM, DRAM, flash, or other memory devices), input/output devices 703, and/or subsystems and other components 704. The computing device 700 can perform any of a wide variety of computing processing, storage, sensing, imaging, and/or other functions. Components of the computing device 700 may be housed in a single unit or distributed over multiple, interconnected units (e.g., though a communications network). The components of the computing device 700 can accordingly include local and/or remote memory storage devices and any of a wide variety of computer-readable media.

As illustrated in FIG. 7, the processor 701 can include a plurality of functional modules 706, such as software modules, for execution by the processor 701. The various implementations of source code (i.e., in a conventional programming language) can be stored on a computer-readable storage medium or can be embodied on a transmission medium in a carrier wave. The modules 706 of the processor can include an input module 708, a database module 710, a process module 712, an output module 714, and, optionally, a display module 716.

In operation, the input module 708 accepts an operator input 719 via the one or more input interfaces associated with the controller 150 described above with respect to FIG. 4, and communicates the accepted information or selections to other components for further processing. The database module 710 organizes records, including patient records, treatment data, treatment profiles and operating records and other operator activities, and facilitates storing and retrieving of these records to and from a data storage device (e.g., internal memory 702, an external database, etc.). Any type of database organization can be utilized, including a flat file system, hierarchical database, relational database, distributed database, etc.

In the illustrated example, the process module 712 can generate control variables based on sensor readings 718 from sensors (e.g., the pressure sensors at 102 and 104 of FIG. 4) and/or other data sources, and the output module 714 can communicate operator input to external computing devices and control variables to the controller 150. The display module 716 can be configured to convert and transmit processing parameters, sensor readings 718, output signals 720, input data, treatment profiles and prescribed operational parameters through one or more connected display devices, such as a display screen, printer, speaker system, etc. A suitable display module 716 may include a video driver that enables the controller 150 to display the sensor readings 718 or other status of treatment progression on an output device (not shown) accessible to a treating physician or surgeon. In a particular embodiment, the display module 716 can include audible or visible alarms (not shown) to communicate patient status, physician intervention requests, disturbances or other abnormalities with patient or system function, etc.

In various embodiments, the processor 701 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 702 can be standard memory, secure memory, or a combination of both memory types. By employing a secure processor and/or secure memory, the system can ensure that data and instructions are both highly secure and sensitive operations such as decryption are shielded from observation.

### E. SUITABLE THERAPEUTIC AGENTS

A therapeutic agent suitable to treat cardiac tissue can be delivered in a targeted manner using the cardiac delivery system 100 of FIGS. 3 and 4 and/or in treatment regimens associated with use of other suitable delivery systems. These therapeutic agent(s) can include a substance that may treat and/or protect biological tissues of the heart of a subject. Examples of therapeutic agents and medical conditions, for which such therapeutic agents can be used for treatment and/or prevention, are described in U.S. Pat. No. 8,556,842, which is incorporated herein by reference in its entirety.

In a particular embodiment, the present technology can be used to conduct targeted delivery of a macromolecular complex used for gene therapy. For example, macromolecular complexes for gene therapy can be useful for treatment of inherited autosomal recessive conditions, such as those associated with the sarcoglycan deficiencies, X-linked cardiomyopathy or the cardiomyopathy associated with Becker's muscular dystrophy. In such embodiments, therapy will involve expression of a missing or dysfunctional gene to correct the particular heart failure phenotype.

Other types of therapies can include, for example, treatment of genetic cardiomyopathies or “idiopathic” heart failure. In addition, the systems and method disclosed herein can be used as an adjunct to valve repair or replacement surgery, coronary artery bypass graft surgery or ventricular assist device (VAD) implantation procedures in selected patients with heart failure. In other embodiments, delivery of therapeutic agents can include the delivery of angiogenic compounds to the heart (and particularly, the myocardium) to treat coronary ischemia. In another example, compounds useful for cancer therapies, including, for example, chemotherapeutic agents useful in treatment of cardiac sarcomas and other neoplasms, can be delivered in a targeted fashion to the cardiac tissue.

In a further embodiment, therapeutic agents can include pharmaceuticals and other chemical agents and small molecules. For example, chemical agents and/or small molecules can include alkylating agents (e.g., cisplatin, carboplatin, streptazoin, melphalan, chlorambucil, carmustine, methclorethamine, lomustine, bisulfan, thiotepa, ifofamide, cyclophosphamide, etc.); hormonal agents (e.g., estramustine, tamoxifen, toremifene, anastrozole, letrazole, etc.); antibiotics (e.g., plicamycin, bleomycin, mitoxantrone, idarubicin, dactinomycin, mitomycin, daunorubicin, etc.); immunomodulators (e.g., interferons, IL-2, BCG, etc.); antimitotic agents (e.g., vinblastine, vincristine, teniposide, vinorelbine, etc.); tipoisomerase inhibitors (e.g., topotecan, irinotecan, etoposide, doxorubicin, etc.); and other agents (e.g., hydroxyurea, traztuzumab, altretamine, retuximab, paclitaxel, docetaxel, L-asparaginase, gemtuzumab, ozogamicin, etc.).

In yet other embodiments, therapeutic agents can be carried to their target tissue by vectors (e.g., plasmids, episomes, cosmids, viral vectors, phage, “naked DNA”) and which can contain a transgene under the control of regulatory sequences. In some embodiments, vectors can carry RNA or DNA molecules, such as modified messenger RNA (mRNA), or other moieties. In certain aspects, therapeutic agents can be macromolecular complexes that can encompass molecules that, due to their large size, are not able to enter a target cell on their own. In additional aspects, therapeutic agents can include molecules that can infect or transfect cells without additional delivery routes.

A selected therapeutic agent can be infused in a physiologically compatible solution prior to delivery. In one embodiment, the solution contains physiologic solution such as, for example, saline, isotonic dextrose, or a glycerol solution, among others that will be apparent to one of skill in the art given the information provided herein. In some embodiments, the physiologic solution can be oxygenated. The concentration of a therapeutic agent in the solution can vary depending upon the type of agent selected. Further, a therapeutic treatment solution may contain more than one therapeutic agents (e.g., two vectors; a vector and a protein, enzyme, or other moiety; or two or more proteins, enzymes, or other moieties).

### F. CLINICAL KITS

The present technology also provides a kit for use by a clinician or other medical personnel and for use with the cardiac delivery system 100 (FIG. 4). In one embodiment, the cannulae, tubing, snares, perfusion solution reservoirs, the perfusion solutions, the therapeutic agent(s), and/or other components (e.g., pumps, switches, valves, oxygenator, PRVC unit, etc.) of the cardiac delivery system 100 can be included in a kit (not shown) for therapeutically implementing a cardiac perfusion circuit for targeted delivery of therapeutics, such as macromolecules, to cardiac tissue of the subject. The kit can also include instruction documentation containing information regarding how to (a) surgically implement the cardiopulmonary bypass procedure to isolate the cardiac circuit from the systemic circuit, and (b) effectively deliver the therapeutic agent(s) to the targeted tissue within the cardiac circuit. In particular embodiments, the kit can include pre-treatment perfusion solutions, therapeutic agent(s) in solution and/or flush solutions in ready to use reservoirs that can be used with the system 100. The kit can further include one or more modules (e.g., software modules) for use with the computing system 700 or other computing device associated with the cardiac delivery system 100.

### G. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A pressure-regulated volume control (PRVC) system for use during cardiopulmonary bypass surgery, comprising:
a PRVC unit configured to regulate a cardiac circuit volume based on a measured return pressure detected at an outflow from an internal heart portion of a cardiac circuit; and
a first pressure sensor configured to detect the measured return pressure.

2. The system of claim 1 wherein the PRVC unit is configured to remove a portion of perfusion solution from a fluid path formed by cardiopulmonary bypass when the measured return pressure exceeds a pre-determined return pressure value.

3. The system of claim 2 wherein the pre-determined return pressure value is about 15 mm Hg.

4. The system of claim 1 wherein the PRVC unit is configured to remove a minimum volume of perfusion solution from a fluid path formed by cardiopulmonary bypass to reduce a return pressure from the measured return pressure to a desired return pressure.

5. The system of claim 1 wherein the PRVC unit is configured to regulate a cardiac circuit volume during a cardiopulmonary bypass procedure on a continuous basis and while a perfusion solution is flowing through a fluid path formed by the cardiopulmonary bypass procedure.

6. The system of claim 1 wherein the PRVC unit regulates a cardiac circuit volume during a cardiopulmonary bypass procedure at intervals and while a perfusion solution is flowing through a fluid path formed by the cardiopulmonary bypass procedure.

7. The system of claim 1, further comprising a controller in communication with the PRVC unit, wherein the controller has instructions for causing the PRVC unit to remove a portion of perfusion volume when the measured return pressure exceeds a maximum allowable return pressure.

8. The system of claim 7, further comprising a second pressure sensor configured to detect a cardiac circuit inflow pressure at an inflow to the internal heart portion of the cardiac circuit.

9. The system of claim 8 wherein the controller has instructions for causing a change to a flow rate of perfusion solution in a fluid path formed by cardiopulmonary bypass based on the detected cardiac circuit inflow pressure.

10. The system of claim 1, further comprising a controller in communication with the PRVC unit and the first pressure sensor, wherein the controller has instructions that are executable to:
(a) receive the measured return pressure from the first pressure sensor in communication with the outflow from the internal heart portion of the cardiac circuit;
(b) calculate a minimum amount of volume of perfusion solution to remove from the cardiac circuit to reduce a return pressure from the measured return pressure to a desired return pressure; and
(c) command the PRVC unit to remove the minimum amount of volume.

11. The system of claim 1 wherein the cardiac circuit has a retrograde fluid path.

12. The system of claim 1 wherein the PRVC unit is configured to regulate the cardiac circuit volume of a perfusion solution comprising a therapeutic agent in a retrograde direction for at least 10 minutes.

13. The system of claim 1 wherein the PRVC unit is configured to remove a minimum volume of the perfusion solution having the therapeutic agent when the measured return pressure exceeds a desired return pressure value.

14. A system for regulating fluid volume from an isolated cardiac circuit during cardiopulmonary bypass surgery, the system comprising:
a controller; and
a pressure-regulated volume control (PRVC) unit in communication with the controller, the PRVC unit configured to regulate a cardiac circuit fluid volume based on a measured return pressure detected at an outflow from an internal heart portion of a cardiac circuit,
wherein the controller has instructions that cause the PRVC unit to—
measure a return pressure at the outflow of the internal heart portion; and
remove a calculated minimum amount of fluid volume from the cardiac circuit.

15. The system of claim 14 wherein the controller has instructions that are executable to:
compare the measured return pressure to a pre-determined maximum return pressure; and
calculate a minimum amount of fluid volume to remove from the cardiac circuit to achieve at least the pre-determined maximum return pressure.

16. The system of claim 14, further comprising a first pressure sensor in communication with the PRVC unit or the controller, the first pressure sensor configured to detect the return pressure at the outflow of the internal heart portion.

17. The system of claim 14 wherein the PRVC unit is configured to remove the calculated minimum amount of fluid volume in an automated manner during cardiopulmonary bypass surgery.

18. A method for removing excess volume of fluid from circulation within a cardiac circuit during cardiopulmonary bypass in a subject, the method comprising:
measuring a return pressure at an outflow of a heart in the subject;
comparing the measured return pressure to a desired return pressure;
calculating an amount of volume of fluid to remove from the cardiac circuit to achieve at least the desired return pressure if the measured return pressure is greater than the desired return pressure; and
removing the calculated amount of volume of fluid.

19. The method of claim 18 wherein removing the calculated amount of volume of fluid includes continuously removing a volume of fluid at a rate of removal between a specified maximum and minimum rate of removal.

20. The method of claim 19, further comprising maintaining the return pressure at or near the desired return pressure by altering the rate of removal.

21. The method of claim 18 wherein removing the calculated amount of volume of fluid includes depositing the fluid in a waste reservoir.

22. The method of claim 21 wherein the total amount of volume of fluid removed from the cardiac circuit does not exceed a predetermined maximum amount of volume of fluid.

23. The method of claim 22 wherein the predetermined maximum amount of volume of fluid is approximately 1000 ml.

24. The method of claim 18 wherein removing the calculated amount of volume of fluid includes manually removing the calculated amount of volume.

25. The method of claim 18 wherein removing the calculated amount of volume of fluid includes removing the volume of fluid in an automated and continuous manner.
